(19) United States
(12) Patent Application Publication
(10) Pub. No.: US 2019/0133807 A1
(43) Pub. Date: May 9, 2019

(54) ORTHOSIS

(71) Applicant: OTTOBOCK SE & CO. KGAA, Duderstadt (DE)

(72) Inventors: Gordon SIEWERT, Gottingen (DE); Tim KRUCHEM, Gottingen (DE)

(73) Assignee: OTTOBOCK SE & CO. KGAA, Duderstadt (DE)

(21) Appl. No.: 16/097,196

(22) PCT Filed: Apr. 26, 2017
(86) PCT No.: PCT/EP2017/059894

§ 371 Date: Oct. 26, 2018

(30) Foreign Application Priority Data
Apr. 29, 2016 (DE) 10 2016 108 049.7

(51) Int. Cl. A61F 5/01

(52) U.S. Cl. CPC A61F 5/0127

(57) ABSTRACT

An orthosis comprising a foot plate for supporting a foot, a foot strut projecting therefrom in the proximal direction, a lower-leg rail secured to said foot strut and mounted thereupon by a joint so as to pivot about a joint axis relative to the foot strut, and a shoulder being arranged or formed at the proximal end region of the foot strut, in the direction facing away from the foot plate.

The invention relates to an orthosis comprising a foot plate for supporting a foot, a foot strut projecting therefrom in the proximal direction, and a lower-leg rail secured to said foot strut and mounted thereupon by a joint so as to pivot about a joint axis relative to the foot strut.

Orthoses are orthopedic devices worn on the body, for example, on a limb or the torso, and which are attached to the respective body part by means of fastening elements, such as straps, cuffs, shoes, or braces. The fastening elements, for example, can be closed by means of clasps or hook and loop fasteners in order to achieve an application on the respective limb adjusted individually to the orthosis wearer. Basically, orthoses are used for assistance, stabilization, relief, support, or also for restriction of motion of the corresponding limbs or joints but also for correcting malalignments, for the correct alignment or retention of a specific orientation as well as for the protection of joints and limbs.

In the area of the lower extremities, orthoses are designed, for example, as knee-ankle-foot orthoses (KAFO) or as ankle-foot orthoses (AFO) and are used, for example, to reduce the effects of a weakening or malfunction of the dorsal flexor muscles. For that purpose, a spring-loaded joint in the area of the natural ankle joint can be provided which preloads the foot plate with regard to the lower-leg rail in order to execute a dorsiflexion in the swing phase, and so a swinging of the foot is still possible without the toes or the forward section of the foot dragging along the floor. Instead of a spring-loaded joint, a spring can also connect a foot plate with the lower-leg rail.

From EP 2 563 300 B1, an orthosis for correcting a leg malalignment is known, comprising a support limb, which grips a foot of a person from underneath, thus creating a contact with a running surface, i.e. either a sole or a floor. The orthosis has a laterally upwardly protruding rail arrangement which can be connected by means of a fastening device with the lower leg of the person, wherein an angle piece, which is rigid under load, is formed at the transition from the support limb to the rail arrangement. The rail arrangement is divided by a swivel joint arranged approximately at the height of the compromise pivot point into an application limb for a lateral application to the foot and a rail provided for a lateral application to the lower leg. The rail is designed as an elastic element; by means of a torque resulting from a set preload of the elastic element relative to the lower leg, a laterally acting corrective force is applied to the lower leg.

The present invention addresses the problem of providing an orthosis which allows for increased wear comfort and an improved guidance of the foot part and an optimized guidance of a lower leg rail.

According to the invention, this problem is solved by an orthosis with the features of the main claim. Advantageous embodiments and developments of the invention are disclosed in the dependent claims, the description, and the drawings.

The orthosis, comprising a foot plate for supporting a foot, a foot strut projecting therefrom in the proximal direction, and a lower-leg rail secured to said foot strut and mounted thereupon by a joint so as to pivot about a joint axis relative to the foot strut, provides that a shoulder is arranged or formed at the proximal end region of the foot strut in the direction facing away from the foot plate. The foot strut, bearing against the bony structure of the foot as closely as possible, extends from the foot plate in proximal direction, i.e. upward, in the direction of the lower leg, and so the foot plate bears against the underside of the foot, and the foot strut bears against the side structure of the foot or can be guided alongside it at close proximity thereto. This results in a very narrow structure in medial-radial direction, and the orthosis can be worn in a shoe with minimal impairment. In order to be able to position the joint as closely as possible in the area, in which the natural ankle joint is located, it is advantageous, when the foot strut, at its proximal end region, has a shoulder which faces in the direction away from the foot, i.e. is arranged or formed in the direction facing away from the foot plate. In case of a medial arrangement of the foot strut, the shoulder is oriented in medial direction; in case of a lateral arrangement of the foot strut, the shoulder is oriented in lateral direction. In case of a medial arrangement of the foot strut, the shoulder is formed by an offset in medial direction in the foot strut, and in case of a lateral arrangement of the foot strut, the shoulder is formed by an offset in lateral direction of the foot strut. As a result, this takes into account that the shinbone and the calf bone form ankle protrusions which make a straight continuation of the foot strut difficult or uncomfortable. The shoulder ensures that the joint and thus also the joint axis can be arranged in the area of the ankle protrusion and as close as possible to the ankle protrusion without impeding movement and enlarging the structure in medial direction. Preferably, the shoulder has a depth between 5 mm and 15 mm and extends over the entire width of the foot strut. The foot strut extends as straight as possible from the foot plate in proximal direction, wherein the shoulder begins below the corresponding ankle protrusion and extends away from the foot, and in turn, subsequently forms a seat for the joint or the joint device in proximal direction. The seat can be designed as a straight section with fastening devices for the joint device.

A development of the invention provides that the joint axis is twistingly oriented about the longitudinal extension of the foot strut, and so, in case of a planar design of the lower foot strut in the area of the linkage to the foot plate, the joint axis is no longer oriented perpendicularly to the foot strut but from a top view is rotated by an angle in longitudinal extension of the foot strut. The rotational or twisting direction is that of a direction of an internal rotation, and so the swivel direction of the joint runs at an acute angle to a foot longitudinal axis or a center line of the foot plate. In an orthosis for a right foot and a medial arrangement of the foot strut, the orientation of the joint axis from a top view onto the foot strut is rotated counterclockwise; in an orthosis for a left foot and a medial arrangement of the foot strut, the orientation of the joint axis is rotated or twisted clockwise.

A development of the invention provides that the shoulder is formed by an offset in the foot strut or an angle piece attached to the foot strut. In case of a one-piece design of the foot strut, the shoulder is obtained by multiple bending of the foot strut. The bending or the offset can be made straight along bending or kinking lines or with enlarged bending radii, and so a kinked or curved bend is achieved.

Alternatively to a one-piece design of the foot strut at least from the foot plate to the proximal end or end region, an angle piece, which has or forms an appropriate offset, can be attached to the foot strut, and so a modular structure of the orthosis can be achieved. A receiving device for the foot strut can be arranged or formed on the angle piece, and so different angle pieces can be placed on a basic model of a foot strut or a foot plate with a foot strut arranged or formed thereon. By means of the angle pieces, the respective orthosis can be easily individualized. Different offset angles, offset orientations, and shoulders can easily be produced within the angle piece, and so an adjustment to different intended purposes and different patients can easily be accomplished. If sturdier joints are used or supporting spring devices are required, larger shoulders can be provided; if only one guide rail is necessary, a narrow shoulder can be selected, and so the structure can be minimized in medial direction.

The foot strut and/or the angle piece are preferably formed from a rigid material in order to be able to transfer a sufficiently great torque between the foot plate and the foot strut. In addition to metal, a fiber-reinforced material can be used for producing the foot strut and/or the angle piece. Provided that a plastic deformability of the material used is present, an additional adaptability, at a simultaneous sufficient rigidity or stiffness, of the orthosis to the respective user can be achieved, for example, by an orthopedic technician. The material is selected such that a sufficient stability is provided after the plastic deformation in order to provide a secure guidance in the frontal plane and the sagittal plane and to furthermore ensure a sufficient torsional stiffness about the longitudinal axis of the foot strut. The strengths and stiffnesses are selected such that in case of a load, a constant angle or at least an essentially constant angle between the foot plate and the foot strut is ensured. The material and its dimensioning must be suitable to be able to transfer a desired torque between the foot plate and the foot strut or the lower leg rail.

At least in its distal region, which is adjacent to the foot plate, the foot strut can extend from the foot plate in proximal direction at an angle between 80° and 100° in order to allow for a preferably even and close application or parallel orientation to the bony structure and the soft-tissue structure of the foot.

The foot strut and possibly the angle piece can be formed from a flat material, thus facilitating easy adjustability for both the orientation and the dimensioning of the shoulder through corresponding deformation.

The joint axis of the joint, which is arranged between the lower leg rail and the foot strut and connects the two components with one another, can have an inclined angle in medial-lateral direction, i.e. from the inner malleolus to the external malleolus in distal direction; in other words, the joint axis extends from the medial side obliquely downward, i.e. slanted laterally downward with regard to the horizontal. Such an orientation of the joint axis corresponds to the natural joint axis of the ankle, and so an improved guidance of the foot is provided by following the natural joint axis.

In addition, it is possible that the joint axis runs obliquely to the foot center line at an angle α, wherein the ankle joint axis, at a medial application of the joint to the foot and the ankle, is positioned in posterior direction. The angle α can be between 70° and 89°. With an assumed orientation of the natural ankle joint axis at a right angle to the foot center line from a top view, i.e. in a horizontal plane in the projection, the joint axis is tilted in anterior direction to the natural ankle joint axis. This can be achieved with a corresponding oblique offset of the foot strut, and so the proximal end region, at which the joint is arranged, is rotated by the desired angle, preferably rotated toward the front between 5° and 20° around the longitudinal extension of the foot strut, particularly between 7° and 15°, as compromise angle by 11°, and so the center axis of the foot or foot longitudinal axis is rotated outwardly with regard to the running direction.

In the applied state, the joint is preferably arranged at the height of the ankle protrusion; at a medial orientation of the foot strut, it is arranged at the height of the medial ankle protrusion; at a lateral orientation of the foot strut, it is arranged at the height of the lateral ankle protrusion. In the applied state, the joint axis is located essentially at the height of the compromise pivot point. A balance in height and orientation can be achieved by a deformation of the foot strut, or by replacing the angle piece, or with the use of distance elements. The compromise pivot point in the ankle joint is the mechanical pivot point of the natural ankle joint which lies below the highest bony point of the medial ankle joint at the height of the lower end of the tibia. From there, the ankle joint axis runs horizontally in a direction lateral to the direction of movement at an angle of 90°.

The orthosis can be worn in a particularly unobtrusive manner, when the foot strut is arranged medially because the medial arrangement of the lower leg rail is very unobtrusive and the bony structure of the foot in the area of the talus and the heel bone additionally allows for a good support of the foot strut.

The foot strut can extend angularly below the foot and have a floor strut which is either molded or fastened to the foot strut. As a result, the floor strut extends below the foot in, on, or below the foot plate and allows for a secure connection of the foot plate with the foot strut.

In addition to a separate design of the foot plate and the foot strut and a securing to one another, it is basically also possible that the foot strut is integral with the foot plate. For that purpose, it is necessary to use the correspondingly suitable materials. In addition to a design made of a medal, it is possible to produce the foot strut and the foot plate as one piece from a composite material, particularly a fiber-reinforced plastic, wherein a seat for the joint or a joint device can be molded on or formed from a different material.

For increasing the wear comfort, the foot plate and/or the foot strut can be provided, at least to some extent, with a sheathing, with which the wear comfort can be improved. In addition, elastic properties of the sheathing can be used for improving the foot roll-over behavior.

In the following, embodiments of the invention shall be explained in more detail using the attached drawings.

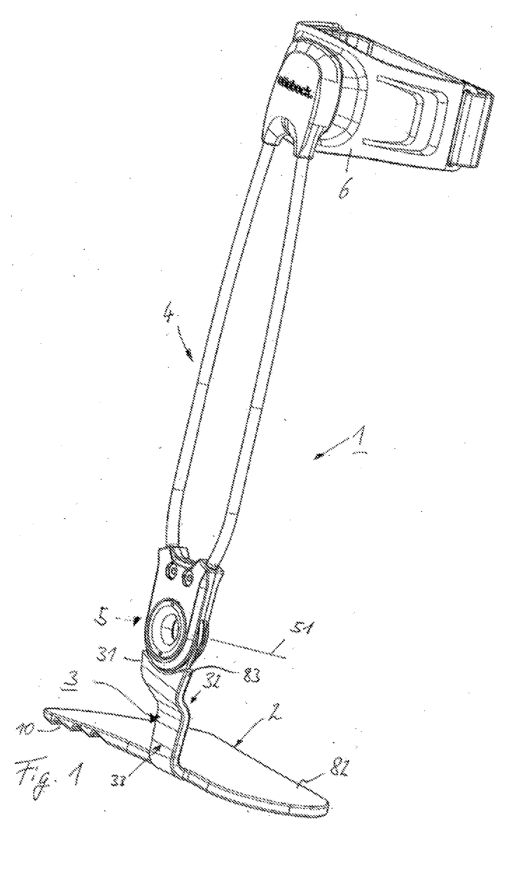

FIG. 1 shows a perspective full view of an orthosis as AFO;

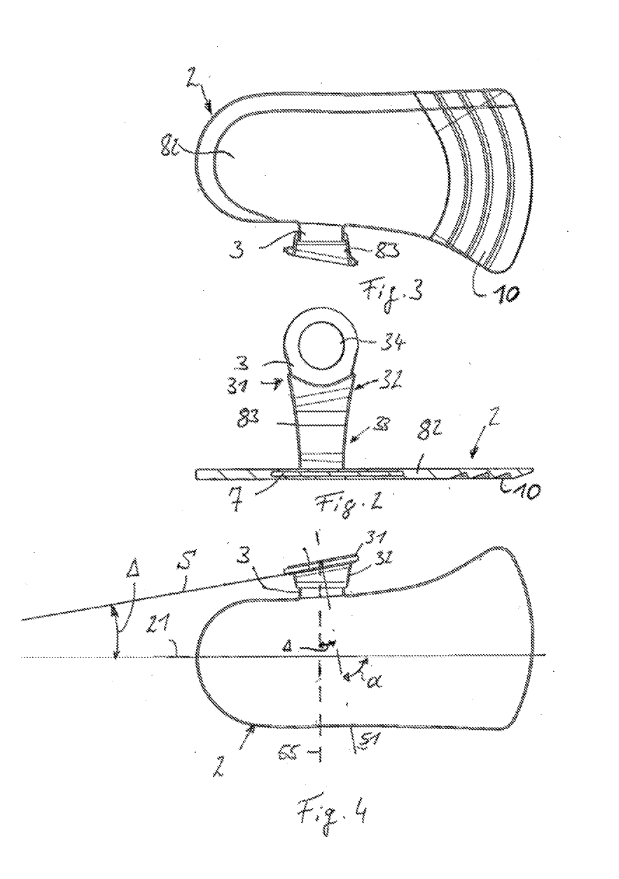

FIG. 2 shows a side view of a foot part of the orthosis without a lower leg rail;

FIG. 3 shows a bottom view of a foot part;

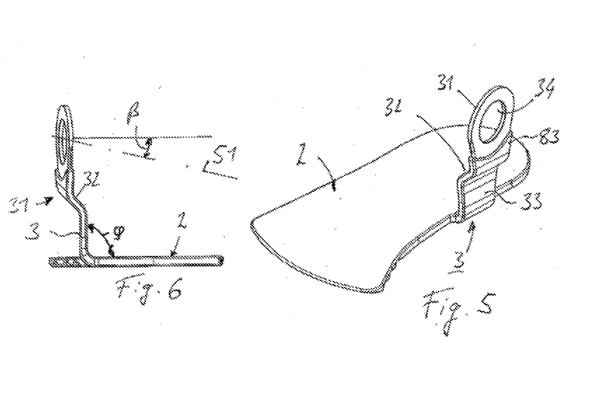

FIG. 4 shows a top view of a foot part;

FIG. 5 shows a perspective diagonal view of a foot part;

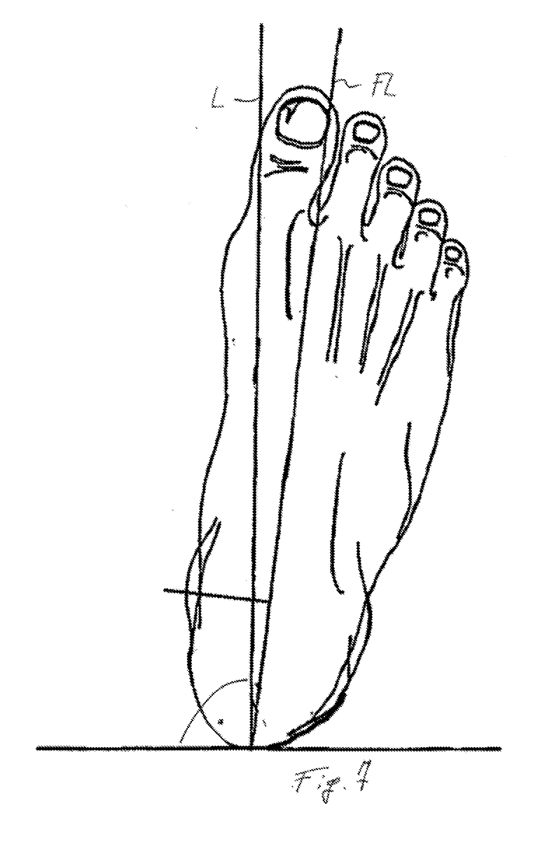

FIG. 6 shows a rear view of a foot part; and

FIG. 7 shows a schematic depiction of a foot.

FIG. 1 shows an orthosis 1 in the form of an ankle-foot orthosis, comprising a foot plate 2 for supporting a foot (not depicted), from which a foot strut 3 projects in proximal direction. At its distal end region 33, the foot strut 3 is fastened to the foot plate 2 or integral with said foot plate 2. The foot strut 3 can be fastened by means of laminating, separate fastening elements, such as screws or rivets, gluing, locking in place or other interlocking or integrally bonding fastening methods. It is also possible that a floor strut 7, which extends in the plane of the floor plate 2 and is shown in FIG. 2, is molded to or fastened to the foot strut 3, said floor strut 7 extending in, on, or below the foot plate 2, thus forming an angle piece that allows for a rigid connection without joints between the foot strut 3 and the foot plate 2.

At the proximal end region 31 of the foot strut 3, a joint 5 is attached which is designed as a flat joint, and by means of which a lower leg rail 4, at the proximal end of which a fastening device 6 is arranged for application on a lower leg (not depicted), is pivotably mounted on the foot strut 3. The fastening device 6 fully encloses the lower leg and has a sleeve with a locking strap. The incline of the upper contact point of the lower leg rail 4 relative to the lower leg or relative to the orientation of the foot strut 3 can be adjusted via two curved struts of the lower leg rail 4. In addition to the depicted orthosis for correcting a leg malalignment, the foot strut 3 can be elastically preloaded with regard to the lower leg rail 4, for example, by means of a spring arranged in the joint 5, in order to provide a drop foot orthosis.

The lower leg rail 4 is pivotable relative to the foot strut 3 about a joint axis 51 via the joint 5; an articulated pivoting about a different axis is not provided.

The foot plate 2 has a sheathing 82 which can be made of a flexible, possibly elastic material. In the depicted embodiment, three consecutively arranged indentations 10 are formed on the underside of the foot plate 2, wherein the footplate 2 can be shortened in a simple manner along said indentations 10.

A sheathing 83 is also arranged on the foot strut 3 which extends to the proximal end 31 of the foot strut 3 but does not cover the joint device 5 and the adjacent area of the proximal end region 31, and so a free pivoting of the lower leg rail relative to the foot strut 3 is not impeded. The sheathing 82, 83 improves the wear comfort because the possibly sharp-edged materials of the foot strut 3 and/or the foot plate 2 cannot come in contact with the body of the orthosis user.

In the proximal end region 31 of the foot strut 3, a shoulder 32 is formed which faces away from the foot plate 2, and so the proximal end region 31 of the foot strut 3 is offset and further away from the foot plate 2 than the distal region 33 of the foot strut 3. Via the shoulder 32, the area, in or at which the joint 5 is arranged, is removed from the foot plate 2, and so the proximal end region 31 is located medially offset to the foot plate 2 at a medial arrangement of the foot strut 3. In case of a lateral arrangement of the foot strut 3, an offset in lateral direction, i.e. away from the foot, is achieved via the shoulder 32. Due to the shoulder 32 and the thus increased distance of the proximal end region 31 of the foot strut 3, when compared to a distal end region 33 of the foot strut, for example, in the region of the transition from the foot plate 2 to the foot strut 3, it is possible that the foot strut 3 can be guided very closely along the bony structure of the foot without the joint 5 rubbing or unpleasantly bearing against the bony ankle protrusion in the area of the ankle.

In the embodiment, the foot strut 3 has a distal end region 33 which extends essentially straight away from the foot plate 2; the distal end region 33 and the proximal end region 31 are, with regard to their sagittal plane, offset to one another due to the shoulder 32. At least the distal end region 33 of the foot strut 3 is formed as a flat material; in the depicted embodiment, the entire foot strut 3 with distal end region 33, shoulder 32, and proximal end region 31 is formed from a flat material, particularly a metal.

FIG. 2 shows the foot part of the orthosis without the lower leg rail 4 and without the joint device with the foot plate 2 which has an essentially planar upper side and an underside with indentations 10 in the front region. The indentations 10 are designed to be sawtooth-shaped and form predetermined interfaces, along which a shortening of the foot plate 2 and thus an adjustment to different shapes of shoes or feet is possible. The indentations 10 have slants which run from the front to the rear in a direction slanted obliquely downward, and so a rolling off is facilitated. If the slant tapers off into a tip, no shoulder is formed at the front edge of the foot plate 2. If possible, a shoulder smaller than 2 mm, preferably smaller than 0.5 mm is desirable in order to prevent impeding influences during the roll-off even in the sensitive foot area. The cross-sectional side view according to FIG. 2 shows the floor strut 7 which, in the depicted embodiment, is formed with the foot strut 3 and constitutes an angle piece without joints. The floor strut 7 is provided with a sheathing 82, and so the foot plate 2 is formed by the floor strut 7 and the sheathing 82. The sheathing 82 also extends over a part of the foot strut 3, where it is denoted with reference sign 83.

The distal section 33 of the foot strut 3 extends essentially vertically upward from the foot plate 2 and is followed by a shoulder 32, in which the material of the foot strut 3 is first bent outward and subsequently bent upward, and so in the proximal end region 31, a joint device, or the bearing for the pivotable mounting of the lower leg rail 4, can be arranged in a seat 34 which, in the depicted embodiment, is designed as a round recess. The proximal end region 31 is designed to be planar and runs essentially in a swivel plane defined by the joint axis 51. The swivel plane stands orthogonally to the joint axis 51 and determines the swivel direction S of the joint. The main plane of the proximal end region 31 lies in the swivel plane or is parallel to it. The swivel plane is essentially oriented in the normal running direction L.

FIG. 3 shows a bottom view of the foot part according to FIG. 2. In the embodiment, the indentations 10 are three indentations 10, run correspondingly to the front edge of the foot plate 2; the foot strut 3 is bent outward from the foot plate 2.

FIG. 4 shows a top view of the foot part with the foot plate 2 and the foot strut 3 projecting perpendicularly from the sheet plane. As in all other depicted embodiments, the foot strut 3 is arranged medially, approximately in the area of the bony ankle protrusion of the shinbone, and extends vertically upward. A minimal bending radius is provided, with which the foot strut 3 is connected to the foot plate 2 via the distal region 33, and so the distal region 33 of the foot strut 3 can bear against the bony structure of the foot as closely as possible.

The shoulder 32 is designed asymmetrically. The front end of the shoulder 32 is bent further toward medial than the rear end, and so from a top view, this results in a swiveling or an angled orientation of the distal end region 31 relative to the implied natural ankle joint axis 55 and relative to a center axis of the foot or a foot center line 21 of the foot plate 2. The angled orientation deviates from a right angle. The joint axis 51 of the joint 5 is aligned oriented at an angle Δ to the natural joint axis and tilted forward, i.e. in an anterior direction, in a horizontal plane. The angle Δ between the natural joint axis 55 and the joint axis 51 of the joint device 5 lies between 5° and 20°, preferably between 7° and 15°, particularly at 12°, in order to compensate an external rotation during walking or standing. Accordingly, the angle Δ between the swivel direction S and the foot center line 21 is between 5° and 20°, preferably between 7° and 15°, particularly at 12°. As seen from the horizontal plane, the joint axis 51 is thus oriented in a slanted manner toward the center line 21 of the foot plate 2 which essentially corresponds to the center line of a foot and runs vertically from the center heel area to the ankle joint axis 55 in anterior-posterior direction. The angle α of the joint axis 51 to the foot center line 21 thus lies between 85° and 70° and faces in anterior direction, i.e. it is oriented rotatingly forward. The foot center line 21 is rotated outwardly at an angle between 20° and 5° relative to the running direction L which corresponds to the swivel direction S.

FIG. 5 shows a perspective diagonal view of the foot part, in which can be seen that the foot strut 3 with the distal section 33 projects essentially vertically from the foot plate 2. As a result of the shoulder 32, the proximal end region 31 with the recess 34 as fastening seat for the joint device 5 is also oriented vertically upward but forwards in longitudinal extension of the foot strut 3 forward or rotated in anterior direction.

FIG. 6 shows a rear view of the foot part. In the depicted embodiment, the angle φ between the foot plate 2 and the distal region of the foot strut 3 is 90° which, in a medial arrangement of the foot strut 3, results in a good application to the foot bone. An individual adjustment is possible by a plastic deformation of the foot strut 3 relative to the foot plate 2, and so the foot strut 3 is applied as closely as possible to the foot. In addition to a rotation about the longitudinal extension in anterior direction, the distal end region 31 of the foot strut 3 can also have an incline, resulting in an angle β between the joint axis 51 and the depicted horizontal. As a result, it is possible that the joint axis 51 of the joint not depicted is individually adjustable. The joint axis 51 is preferably positioned such that it coincides with the natural ankle joint axis. With an angle adjustment of the foot strut 3, a change of the shoulder 32, by means of distance elements, or insertions or attachments, when the lower leg rail 4 is attached, the height can be adjusted. In its applied state, the joint axis 51 is positioned approximately, preferably exactly, at the height of the compromise pivot point of the natural ankle joint.

FIG. 7 shows a schematic depiction of a foot, in which the running direction L and the longitudinal axis FL of the foot are indicated. At a normal foot position, the running direction L and the longitudinal axis FL of the foot diverge; as a rule, the longitudinal axis FL of the foot is oriented externally rotated with regard to the running direction L.

1. An orthosis comprising:
a foot plate configured to support a foot;
a foot strut projecting from the foot plate in a proximal direction;
a lower-leg rail secured to said foot strut and mounted thereupon by a joint so as to pivot about a joint axis relative to the foot strut;
a shoulder is arranged or formed at a proximal end region of the foot strut, in a direction facing away from the foot plate.

2. The orthosis according to claim 1, wherein the joint axis is twistingly oriented to a longitudinal extension of the foot strut.

3. The orthosis according to claim 1, wherein the shoulder is formed by an offset in the foot strut or an angle piece fastened to the foot strut.

4. The orthosis according to claim 3, wherein the foot strut or the angle piece are formed from a rigid material.

5. The orthosis according to claim 1, wherein the foot strut extends from the foot plate in the proximal direction at an angle between 80° and 100°.

6. The orthosis according to claim 1, wherein the foot strut is formed from a flat material.

7. The orthosis according to claim 1, wherein the joint axis in medial-lateral direction is slanted at an angle in a distal direction.

8. The orthosis according to claim 1, wherein the joint axis runs obliquely to a center line of the foot plate at an angle.

9. The orthosis according to claim 8, wherein in a applied state, the joint axis defines a swivel direction which is oriented at an angle between 5° and 20° to a longitudinal axis of the foot or the center line of the foot plate.

10. The orthosis according to claim 9, wherein in the applied state, the joint is arranged at a height of a compromise pivot point.

11. The orthosis according to claim 1, wherein the foot strut is arranged medially on the foot plate.

12. The orthosis according to claim 1, further comprising a floor strut is molded or fastened to the foot strut and extending into or below the foot plate.

13. The orthosis according to claim 1, wherein the foot strut is integral with the foot plate.

14. The orthosis according to claim 1, wherein at least one of the foot plate and the foot strut are provided with a sheathing.

15. An orthosis comprising:
a foot plate configured to support a foot;
a foot strut projecting from the foot plate in a proximal direction;
a lower-leg rail pivotally connected to the foot strut with a joint, the joint providing pivotal movement of the lower-leg rail relative to the foot strut;
a shoulder positioned at a proximal end region of the foot strut and arranged in a direction facing away from the foot plate.

16. The orthosis according to claim 15, wherein a rotation axis of the joint is oriented to a longitudinal extension of the foot strut.

17. The orthosis according to claim 15, wherein the shoulder is formed by an offset in the foot strut or an angle piece fastened to the foot strut.

18. The orthosis according to claim 17, wherein the foot strut or the angle piece are formed from a rigid material.

19. The orthosis according to claim 15, wherein the foot strut extends from the foot plate in the proximal direction at an angle between 80° and 100°.

20. The orthosis according to claim 1, wherein the foot strut is formed from a flat material.
